(19) United States
(12) Patent Application Publication
(10) Pub. No.: US 2023/0157310 A1
(43) Pub. Date: May 25, 2023

(54) METHOD TO FORMULATE AND PRODUCE VEGETABLE OIL BLENDS CONTAINING OPTIMALLY BALANCED FATTY ACIDS

(71) Applicant: Sudhir P. Deosarkar, Methuen, MA (US)

(72) Inventor: Sudhir P. Deosarkar, Methuen, MA (US)

(21) Appl. No.: 18/056,482

(22) Filed: Nov. 17, 2022

Related U.S. Application Data
(60) Provisional application No. 63/264,365, filed on Nov. 21, 2021.

(51) Int. Cl. A23D 9/04; A61K 31/201; A23L 33/12

(52) U.S. Cl. CPC A23D 9/04; A61K 31/201; A23L 33/12; A23V 2002/00

(57) ABSTRACT

Modern diet has a very high omega 6 fatty acid content and a very high omega 6:omega 3 fatty acid ratio. The major sources of these in the modern diet are the vegetable oils consumed today. This imbalance of omega 6 and omega 3 fatty acids and the excessive amounts of omega 6 fatty acids in today's diet have been implicated in several chronic diseases. Thus, improving the balance of these fatty acids in the diet can have a positive nutritional and health impact. The present invention provides a method for formulating and producing vegetable oil blends, such that these vegetable oil blends will contain an optimal balance and optimal amounts of omega 6 and omega 3 fatty acids, along with optimal amounts of other oil nutrients, thus providing a vast improvement in the nutritional profile compared to the currently available vegetable oils.

### FIELD OF THE INVENTION

The present invention relates to a method that enables formulation and production of one or more or several blends of vegetable oils that are uniquely formulated by blending certain preferred and/or select vegetable oils in precise amounts such that the resulting blends will provide key fatty acids and other nutrients in optimally balanced amounts and ratios for the purposes of this invention. More particularly, the present invention provides a method and process for formulating vegetable oil blends by combining precise amounts of one or more High Omega 3 (ω3) fatty acid containing Vegetable Oil (hereafter denoted as HOVO), such as including but not limited to, flaxseed oil, with precise amounts of one or more of the low omega 3 (ω3) and/or high omega 6 (ω6) fatty acid containing vegetable oils, and/or vegetable oil(s) containing none or very low omega 3 (ω3) and/or omega 6 (ω6) fatty acids, selected from, including but not limited to, vegetable oils such as olive oil, coconut oil, avocado oil, safflower oil, rapeseed (canola) oil, sunflower oil, peanut oil, sesame oil, corn oil, soybean oil, cottonseed oil, grapeseed oil, walnut oil and combinations thereof, such that the resulting oil blend provides omega 6:omega 3 (ω6:ω3) fatty acid ratio, including but not limited to, of 1:1, 1:2, 2:1, 1.5:1, 1:1.5 etc., with ω6 and ω3 fatty acids each in optimal amounts, including but not limited to, any amount ranging from 100 mg to 3000 mg per tablespoon of the blend. Additionally, the vegetable oil blends produced using the present invention will provide a significant improvement in the nutritional profile and potential health benefits over the vegetable oils and vegetable oil blends available on the market today, as well as this invention may also provide improvements in other physico-chemical properties of vegetable oils such as, including but not limited to, an improved and/or different flavor profile.

### BACKGROUND OF THE INVENTION AND THE PROBLEM STATEMENT

The following description of the background of the invention is provided simply as an aid in understanding the invention and is not admitted to describe or constitute prior art to the invention.

Vegetable oils are an important part of daily human diet. The use of vegetable oils in diet has evolved significantly over the last several decades and have gained more prominence in the recent years. The use of vegetable oils (or fats) over animal fats has become more popular in the recent years due to the several nutritional and health benefits offered by the vegetable oils over animal fats. There has been significant research in recent years on both the benefits and the deleterious effects of various types of fats, oils and fatty acids on human health. As a result, today we have a much better understanding of the vegetable oils that we consume in terms of their composition and nutrition as well as their potential effects on health and disease. From this research, it is becoming more and more evident that, although several vegetable oils in the market today offer certain important nutrients and favorable health benefits, a vegetable oil from a single source may not offer the right balance of all the important nutrients, thus it may not offer proper health benefits, and in certain circumstances may even be harmful to health.

Today's market is flooded with many choices of vegetable oils for human consumption. These include olive oil, coconut oil, avocado oil, safflower oil, rapeseed (canola) oil, sunflower oil, peanut oil, sesame oil, corn oil, soybean oil, cottonseed oil, grapeseed oil, walnut oil, and so forth. A lot of them claim health benefits over other sources of fats (such as animal fats) and fatty acids in the diet. Although, this is true in some cases, when looking at certain individual elements of a given vegetable oil, a deeper analysis of the compositions of the vegetable oils carried out by various studies in the last several years tells a somewhat different story.

A couple of particular aspects of the vegetable oils, currently available on the market, that deserve a careful examination are (1) the amount of certain polyunsaturated fatty acids (PUFAs), i.e., omega6 (ω6) and omega 3 (ω3) fatty acids (some are in excess while the others are either absent or present in miniscule amounts), and (2) the balance of these fatty acids with respect to each other (i.e. the ratio of one over the other) is abnormal. Historically, a vegetable oil that provided PUFAs and monounsaturated fatty acids (MUFAs), i.e., omega 3 (ω3), omega 6 (ω6) and omega 9 (ω9) fatty acids, in sufficient quantities has been considered beneficial for overall health. In fact, omega 3 and omega 6 fatty acids are essential fatty acids as they cannot be produced by the human body, thus they need to be supplemented through diet, whereas omega 9 fatty acids can be produced by the human body and thus are although not essential, a diet supplemented with omega 9 fatty acid has shown positive impact on health. Although, the presence of these fatty acids in diet is essential for maintaining optimal health, recent research has found that, in particular, the excessive consumption of ω6 fatty acids and an improper balance of ω6 fatty acid with other fatty acids, including with ω3 fatty acids, in the diet can in fact have deleterious effects on health and may lead to chronic diseases. Some of such research is discussed below.

As discussed above, omega 6 fatty acid {i.e., linoleic acid (LA)} is an essential fatty acid, but excessive consumption of this fatty acid has been linked to many chronic diseases such as obesity, diabetes, and cardiovascular diseases potentially leading to premature morbidity and mortality. Alvheim et al. have shown that dietary linoleic acid (LA) increases the key obesity markers 2-arachidonoylglycerol (2_AG) and Anandamide (AEA), thus leading to obesity initiation and progression (Alvheim et al., 2012). In another study, Kain et al. found that excessive consumption of ω6 fatty acids causes metabolic defects in young and also in ageing mice which leads to chronic inflammation (Kain et al., 2018). Luo et al., in their study, show that excess consumption of ω6 fatty acids leads to sleep disorders (Luo et al., 2021). Another study by Weir et al., showed that there is a strong association between ω6 fatty acids and hyperinsulinemia which can lead to diabetes (Weir et al., 2021). Additionally, a strong correlation was found between consumption of soybean oil containing high ω6 fatty acids and obesity inducing markers in mice, noting that soybean oil also has a high ω6:ω3 fatty acid ratio and high omega 6 content (Deol et al., 2017).

Similar to ω6 fatty acids, ω3 fatty acids {i.e., alpha-linoleic acid (ALA), eicosapentaenoic acid (EPA) and docosahexaenoic acid (DHA)} are also essential fatty acids. But in contrast to ω6 fatty acids, ω3 fatty acids appear to show more favorable health benefits. This may in part be due to the fact that modern diet has significantly higher ω6 fatty acids than ω3 fatty acids and also in part due to the fact that ω3 fatty acids show health benefits when consumed in dietary source or as a dietary supplement and there are no reports of any significant deleterious effects of excessive consumption of ω3 fatty acids. As studied by Gutierrez et al., ω3 fatty acids play an important role in immune cells and immune cell function, thus they regulate cellular function and can ameliorate symptoms in various inflammatory, infectious, autoimmune and other chronic diseases (Gutierrez et al., 2019). A metanalysis of literature on the effects of ω3 fatty acids on cardiovascular diseases suggests that increased consumption of ω3 fatty acids may reduce the risk of cardiovascular disease events (Abdelhamid et al., 2020). Similar cardiovascular benefits, along with positive benefits on various other diseases, were observed by Djuricic and Calder with respect to dietary ω3 fatty acids (Djuricic and Calder, 2021).

As opposed to ω3 and ω6 fatty acids, ω9 fatty acids {i.e., oleic acid (OA)} are not essential fatty acids as human body is able to produce them if they cannot be obtained through diet. ω9 fatty acids appear to possess antioxidant and anti-inflammatory properties similar to ω3 and ω6 fatty acids, except excessive consumption of ω9 fatty acids (and ω3 fatty acids) do not appear to be deleterious to health. In fact, ω9 fatty acids provide several health benefits, including their role in the development of several tissues and organs including the brain. One study showed that ω9 fatty acid is able to reduce inflammation in experimental sepsis, thus suggesting that ω9 fatty acid could be used to either treat sepsis or at least reduce the time to recovery from sepsis (Medeiros-De-Moraes et al., 2018). Another study looked at the effect of ω9 fatty acid on pain receptor TRPV1 and found that it acts as an inhibitor of TRPV1 thus provides a basis for potentially treating various neurological and skin conditions that involve pain and itch (Morales-Lazar® et al., 2016). Yet another study shows the benefits of dietary ω9 fatty acids on preventing the development of inflammatory and cardiovascular diseases (Harvey et al., 2010).

Based on the above research, it is reasonable to conclude that ω3, ω6 and ω9 fatty acids in diet offer several health benefits and in fact provide a basis for potentially treating or preventing various chronic diseases, but also excessive consumption of ω6 fatty acids and/or very high ω6:ω3 fatty acid ratio can lead to the development of the very diseases that they may be able to prevent or treat if consumed in moderation and in the right optimal balance. Various studies, including the studies discussed above, have suggested that a diet that has very high ω6 fatty acid content and/or very high ω6:ω3 fatty acid ratios can lead to the development and progression of several chronic diseases (Blasbalg et al., 2011; Djuricic and Calder, 2021; Simopoulos, 2002; Simopoulos, 2016). This suggests that the presence of ω3 fatty acids in diet is beneficial and moderate amounts of ω6 fatty acids and an optimally balanced ω6:ω3 fatty acid ratio reduces the incidence of chronic diseases and is in fact advantageous to maintaining an optimal health. Thus, it is clear that when the ratio of ω6:ω3 fatty acids is much higher in diet, as is the case for the modern diet (especially the western diet and some modern eastern diet), it can have significant deleterious effects on health. Historically, a typical diet that provided a ω6:ω3 ratio of 1:1 or at the most 2:1, has shown a good correlation with an optimal health, as observed for the pre-industrial period (Simopoulos, 2016). Since vegetable oils are a major part of the modern diet, it would be advantageous to have vegetable oils for consumption that provide a better balance of ω6 and ω3 fatty acids and a better ω6:ω3 fatty acid ratio.

A closer look at the composition of the vegetable oils available on the market today reveals that these vegetable oils provide ω6 and ω3 fatty acids in a ratio that is much higher than 1:1 and 2:1 (ω6:ω3). In fact, the only vegetable oil that provides the ω6:ω3 fatty acid ratio close to 2:1 is rapeseed (canola) oil and the other vegetable oils available today have a much higher ω6:ω3 fatty acid ratio. One thing to note about canola oil is that, although it provides a ω6:ω3 ratio of 2:1, the quantity of ω6 fatty acid, about 2.6 g per 14 g (or 1 tablespoon) of oil {ω3 fatty acid is about 1.28 g per 14 g (or 1 tablespoon) of canola oil}, is not insignificant compared to the other vegetable oils {e.g., olive oil has 1370 mg of ω6 fatty acids per 14 g (or 1 tablespoon) of oil}. Majority of the other vegetable oils available on the market have ω6:ω3 fatty acid ratio that is much higher than 1:1 and 2:1. For example, olive oil on average provides about 1370 mg of ω6 fatty acids per 14 g (or 1 tablespoon) of oil but has a ω6:ω3 ratio of 12:1, avocado oil on average provides about 1750 mg of ω6 fatty acids per 14 g (or 1 tablespoon) of oil but has an ω6:ω3 ratio of 13:1 and corn oil on average provides about 7490 mg of ω6 fatty acids per 14 g (or 1 tablespoon) of oil and has an ω6:ω3 ratio of 45:1. As we can see from these examples, the majority of the vegetable oils available on the market for consumption today provide ω6 fatty acids in much more excess and/or have very high ω6:ω3 fatty acid ratios.

In addition to the vegetable oils, several other dietary sources, e.g., nuts, nut derived products, certain seeds, and oil supplements (such as “Essential Balance Oil Blend” by Omega Nutrition company and other PUFA oil supplements), can also introduce larger and larger amounts of ω6 fatty acids in the diet. Thus, even with the use of oils with relatively moderate ω6:ω3 ratio (e.g., canola oil, olive oil, etc.), it would be very difficult to maintain a moderate level of consumption of overall ω6 fatty acids in the diet and maintain an optimal ω6:ω3 fatty acid ratio. Since vegetable oils are a major part of today's diet, they are the major source of the ω6 and ω3 fatty acid imbalances.

In the recent years, there have been a few attempts at blending certain vegetable oils in order to provide consumers options of using vegetable oil blends. But these blends have been randomly formulated without much attention to how this blending affects the final blend composition. Certain such blends have provided some beneficial improvements over pure vegetable oils from a single vegetable (i.e., plant) source but these blends provide only marginal improvements and do not solve the problems associated with high ω6 fatty acids and high ω6:ω3 fatty acid ratio. For example, Crisco, a commercial entity, provides a vegetable oil blend of canola, sunflower and soybean oil; Wesson has a vegetable oil blend of soybean oil and canola oil; SmartBalance, a commercial entity, has a vegetable oil blend of canola, soybean and olive. None of these and other available blends provide anything more than a marginal improvement in the vegetable oil characteristics and none of them have a better ω6 fatty acid profile or ω6:ω3 fatty acid ratios. One vegetable oil blend by Omega Nutrition, a commercial entity, has an oil blend on the market named “Essential Balance Oil Blend”, that is available as a dietary supplement and not as a vegetable oil blend for cooking and food preparations, that claims to contains ω6:ω3:ω9 fatty acid ratio of 1:1:1, but this is misleading as the individual quantities of each of these fatty acids in one tablespoon exceed 4000 mg (1390 mg per teaspoon), which is a very high ω6 fatty acid content compared to even some of the pure vegetable oils such as olive oil, avocado oil, canola oil, and flaxseed oil, which carry about 1370 mg, 1750 mg, 2400 mg and 2000 mg of ω6 fatty acids respectively in one tablespoon. Thus, this “Essential Balance Oil Blend” will not at all be suitable for everyday dietary needs (such as cooking, baking, sauteing, dressing, or any other food preparations or similar uses); plus even if a vegetable oil is to be used as a nutritional supplement, it would be preferred to have a vegetable oil blend that provides healthy ω6:ω3 fatty acid ratios (and possibly including healthy ω6:ω3:ω9 fatty acid ratios) along with healthy and optimal amounts of individual fatty acids (especially ω6 fatty acids) per volume/amount (e.g. per tablespoon) of the vegetable oil blend. As we can see, all of these and similar vegetable oil blends available on the market today will still pose the same nutritional and health challenges that are discussed above with respect to the vegetable oils. Thus, the present invention overcomes these challenges and provides a method to precisely formulate and produce such preferred superior vegetable oil blends that currently do not exist on the market or elsewhere.

### The Solution the Present Invention Provides

The present invention addresses these above challenges by providing a method of blending precise amounts of one or more High Omega 3 (ω3) fatty acid containing Vegetable Oil (HOVO), such as including but not limited to, flaxseed oil, with precise amounts of one or more of the low omega 3 (ω3) and/or high omega 6 (ω6) fatty acid containing vegetable oils, and/or vegetable oil(s) containing none or very low omega 3 (ω3) and/or omega 6 (ω6) fatty acids, selected from, including but not limited to, vegetable oils such as olive oil, coconut oil, avocado oil, safflower oil, rapeseed (canola) oil, sunflower oil, peanut oil, sesame oil, corn oil, soybean oil, cottonseed oil, grapeseed oil, walnut oil and combinations thereof, such that the resulting oil blend provides omega 6:omega 3 (ω6:ω3) fatty acid ratio, including but not limited to, of 1:1, 1:2, 2:1, 1.5:1, 1:1.5 etc., with ω6 and ω3 fatty acids each in optimal amounts, including but not limited to, any amount ranging from 100 mg to 3000 mg per tablespoon of the blend. Additionally, the vegetable oil blends produced using the present invention will provide a significant improvement in the nutritional profile and potential health benefits in the resulting blends over the vegetable oils and vegetable oil blends available on the market today, as well as this invention may also provide improvements in other physico-chemical properties of the vegetable oils such as, including but not limited to, an improved and/or different flavor profile. Thus, this invention provides a method to formulate and produce vegetable oil blends with optimally balanced fatty acids such that the ω6:ω3 fatty acid ratio and the amounts of each of these fatty acids, including but not limited to, such as ω6, ω3, ω9 fatty acids, can be controlled and modified as described in this invention to provide superior vegetable oil blends with superior fatty acid profiles, quantities and ratios.

### Definitions

As used herein, the terms “fat’, “oil”, “vegetable oil” and “fatty acids” are used interchangeably to refer to an edible triglyceride-based composition and other fat components. Such fats and oils can be obtained from a variety of vegetable/plant sources, such as plants, vegetables, and their individual components including the seeds, fruit, or leaves. Fats are solid or semi-solid at room temperature, while oils are generally fluid at room temperature. As used herein, ω6 fatty acids can mean one or more of the fatty acids from the list including linoleic acid (LA), gamma-linoleic acid (GLA), calendic acid, eicosadienoic acid, dihomo-gamma-linolenic acid (DGLA), arachidonic acid (AA, ARA), etc. of the omega 6 fatty acid class. As used herein, ω3 fatty acids can mean one or more of the fatty acids from the list including alpha-linoleic acid (ALA), eicosapentaenoic acid (EPA), docosahexaenoic acid (DHA), etc. of the omega 3 fatty acid class. As used herein, ω9 fatty acids can mean one or more of the fatty acids from the list including oleic acid (OA), elaidic acid, etc. of the omega 9 fatty acid class. As used herein, HOVO means High Omega 3 (ω3) fatty acid containing Vegetable Oil which includes any one or more oils such as, including but not limited to, flaxseed oil, chia seed oil, algal oil, hemp seed oil etc. and so on.

### BRIEF SUMMARY OF THE INVENTION

Modern diet, especially the western diet, has a very high omega 6 fatty acid content and a very high omega 6:omega 3 fatty acid ratio. The major source of these in the modern diet are the vegetable oils consumed today, most of which have a very high omega 6:omega 3 fatty acid ratio and a very high omega 6 content, including the various vegetable oil blends available on the market today. This imbalance of omega 6 and omega 3 fatty acids and the excessive amounts of omega 6 fatty acids in today's diet have been implicated in several chronic diseases. Thus, improving the balance of these fatty acids in the diet can have a positive nutritional and health impact. The present invention provides a method for formulating and producing vegetable oil blends, such that these vegetable oil blends will contain an optimal balance and optimal amounts of omega 6 and omega 3 fatty acids, along with other oil nutrients. The vegetable oil blends, as formulated and produced in the present invention, will provide a vast improvement in the nutritional and health characteristics for consumers over the vegetable oils and vegetable oil blends currently available on the market. Additionally, the vegetable oil blends produced using the present invention may also provide improvements in other physico-chemical properties of vegetable oils such as, including but not limited to, an improved and/or different flavor profile.

### BRIEF DESCRIPTION OF THE DRAWINGS

FIG. 1: A flowchart depicting the exemplary steps in the method and process of formulating and producing vegetable oil blends described in the present invention. This method or process has several steps indicated by numerals from 100 to 107, including additional dependent parts of each step denoted by the main numeral followed by a letter (e.g., 100a, 101a, and so on). The order and contents of these steps are as shown in this figure are for demonstration purpose and they are not meant to limit the sequence and content of the steps, the order and the content of the steps may be changed if necessary to achieve the objectives of the present invention.

FIG. 2A: An exemplary Microsoft Excel worksheet showing known and unknown values of the variables used in the equations (1) through (5) as well as calculations and simulations of these equations for the determination of vegetable oil blend compositions, all of these are indicated by the numerals from 108 to 117, including additional dependent parts denoted by the main numeral followed by a letter (e.g., 117a, and so on). This exemplary worksheet shows vegetable oil blends formulated using flaxseed oil, olive oil, coconut oil and avocado oil. This exemplary worksheet shows values for various parameters used in the determination of the “Example Vegetable Oil Blend 1” below. This worksheet is only exemplary and should not be interpreted as limiting, and in fact, using this methodology, similar worksheets can be built for other combinations and permutations of the vegetable oils shown in the worksheet as well as several other vegetable oils as described in the present invention, including but not limited to, Example Vegetable Oil Blends 2-16.

FIG. 2B: Screenshot of the Microsoft Excel GRG Nonlinear Solver along with exemplary solver parameters used in solving equations (1) through (5) using the exemplary Microsoft Excel worksheet shown in FIG. 2A. Various elements of the solver are indicated by the numerals from 118 to 122, including additional dependent parts denoted by the main numeral followed by a letter (e.g., 120a). The constraints and other parameters shown in FIG. 2B are for the “Example Vegetable Oil Blend 1” used here for demonstration purpose and should not be interpreted as limiting, and in fact, using this methodology, similar calculations/simulations can be built/performed for other combinations and permutations of the vegetable oils shown in the worksheet in FIG. 2A as well as several other vegetable oils as described in the present invention, including but not limited to, Example Vegetable Oil Blends 2-16.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific details disclosed herein are not to be interpreted as limiting, but merely as a basis for the claims and as a representative basis for teaching one skilled in the art to variously employ the present invention in virtually any appropriate manner.

As discussed in the background section above, the vegetable oils and the vegetable oil blends available today (available on the market or as described in the literature) lack a proper balance of fatty acids (including that of ω6 and ω3 fatty acids) and/or do not provide certain fatty acids in properly balanced amounts (i.e. some vegetable oils either do not provide certain fatty acids in sufficient quantities or provide some other fatty acids in too much excess), which as shown by several studies in the literature, may lead to or contribute to the incidence or progression of certain chronic diseases. The present invention solves this problem as described in detail below.

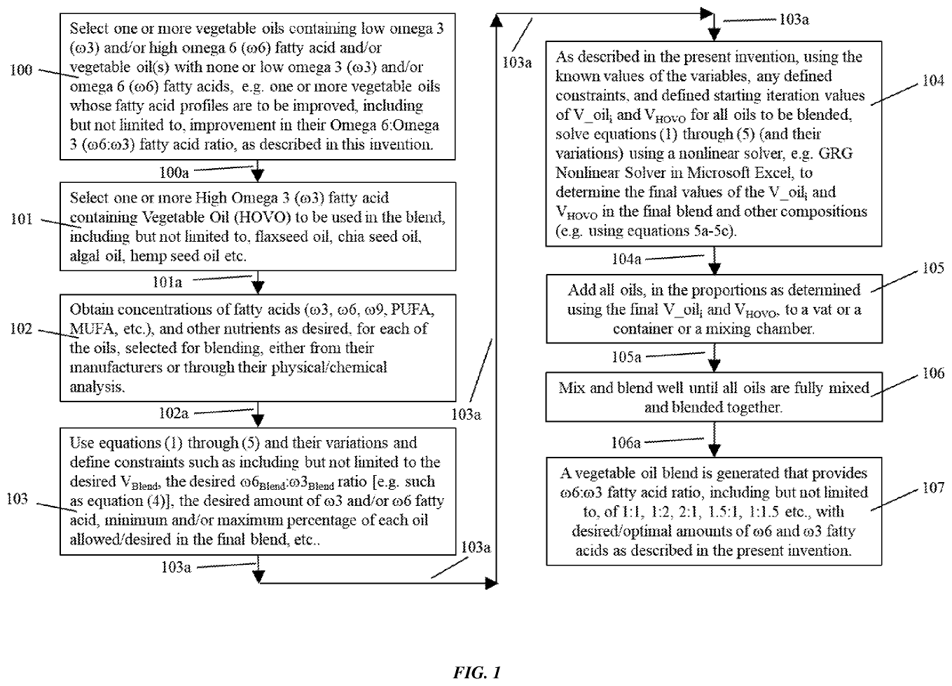

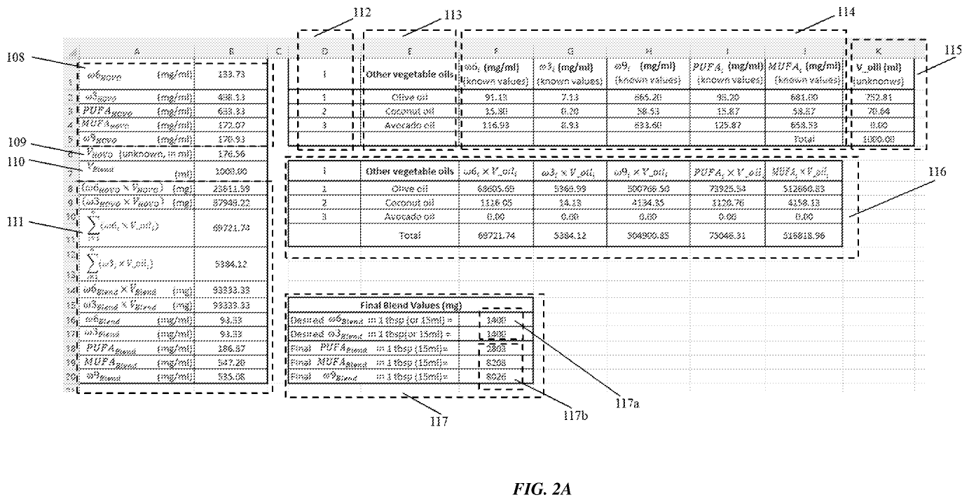

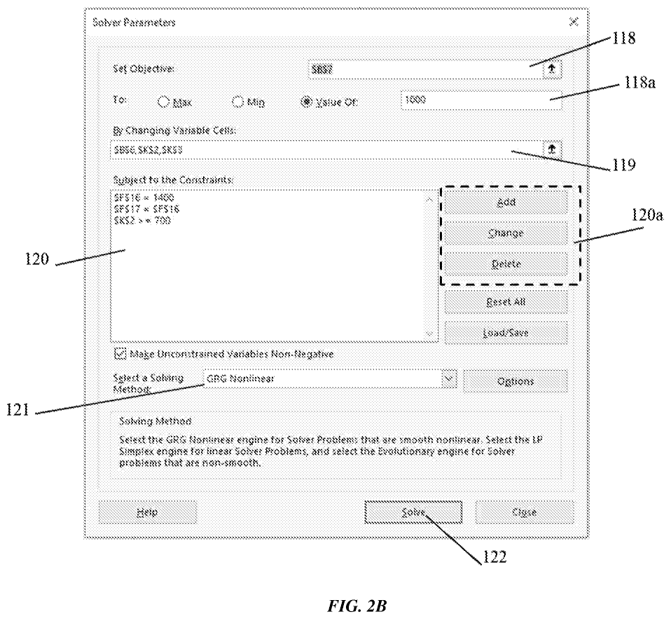

The present invention describes a method that the inventor has developed wherein by combining one or more High Omega 3 (ω3) fatty acid containing Vegetable Oil (HOVO), such as, not limited to but including, flaxseed oil, in certain precise proportions with one or more of the low omega 3 (ω3) and/or high omega 6 (ω6) fatty acid containing vegetable oils, and/or vegetable oil(s) containing none or very low omega 3 (ω3) and/or omega 6 (ω6) fatty acids, selected from olive oil, coconut oil, avocado oil, safflower oil, rapeseed (canola) oil, sunflower oil, peanut oil, sesame oil, corn oil, soybean oil, cottonseed oil, grapeseed oil, and combinations thereof, that will generate vegetable oil blend(s) that will provide ω6: ω3 fatty acid ratio, not limited to but including, of 1:1, 1:2, 2:1, 1.5:1, 1:1.5 etc., with ω6 and ω3 fatty acids in the desirable amounts, including but not limited to, each ranging from 100 mg to 3000 mg per tablespoon of the blend, and thus such vegetable oil blends will provide a significant improvement in the nutritional profile and potential health benefits over the vegetable oils available on the market today. FIG. 1 shows the steps 100 through 107, including additional dependent parts (e.g., 100a, 101a, and so on), in the method developed and described in the present invention. The order and content of these steps is as shown in this figure (FIG. 1) for demonstration purpose and is not meant to limit the sequence and content of the steps, these may be changed or modified if necessary to achieve the objectives of the present invention. FIGS. 2A and 2B show exemplary demonstration of how to solve the equations developed in this invention for obtaining the vegetable oils and vegetable oil blends of the present invention. The worksheet, calculations and simulations depicted in FIGS. 2A and 2B are only exemplary and should not be interpreted as limiting, and in fact, using this methodology, similar worksheets, calculations and simulations can be built for other combinations and permutations of the vegetable oils shown in the worksheet as well as several other vegetable oils as described in the present invention, including but not limited to, Example Vegetable Oil Blends 2-16.

For the purpose of formulating vegetable oil blends that will provide ω6:ω3 fatty acid ratio, not limited to but including, of 1:1, 1:2, 2:1, 1.5:1, 1:1.5 etc., with ω6 and ω3 fatty acids each in the desirable amounts, a set of the following equations {equations (1) through (5) and their other variations as illustrated below}, and a way of solving these equations to obtain desired vegetable oil blend compositions, is developed in this invention as described below.

(ω6_{HOVO}×V_{HOVO})+Σᵢ₌₁ⁿ(ω6ᵢ×V_oilᵢ)=ω6_{Blend}×V_{Blend} (1)

(ω3_{HOVO}×V_{HOVO})+Σᵢ₌₁ⁿ(ω3ᵢλV_oilᵢ)=ω3_{Blend}×V_{Blend} (2)

V_{Blend}=V_{HOVO}+Σᵢ₌₁ⁿ(V_oilᵢ) (3)

These equations and their parts are embedded in certain worksheet cells of parts 111, 116 and 117 of FIG. 2A. Wherein,

ω6_{HOVO} is the amount of omega 6 fatty acid content of High Omega 3 fatty acid containing Vegetable Oil (HOVO), such as flaxseed oil, in mg per ml.

ω3_{HOVO} is the amount of omega 3 fatty acid content of High Omega 3 fatty acid containing Vegetable Oil (HOVO), such as flaxseed oil, in mg per ml.

ω6, is the amount of omega 6 fatty acid content of vegetable oil i, that is to be blended with HOVO, in mg per ml.

ω3, is the amount of omega 3 fatty acid content of vegetable oil i, that is to be blended with HOVO, in mg per ml.

V_{HOVO} is the volume of the High Omega 3 fatty acid containing Vegetable Oil (HOVO), such as flaxseed oil, in the blend in ml.

V_oilᵢ is the volume of each oil i, that is to be blended with HOVO, in the blend in ml.

i is the number from 1 to n, each representing separate pure or individual vegetable oil (derived from a single plant or vegetable or any of their parts) that is to be blended with HOVO (e.g. as shown in FIG. 2A, part 112).

n is the total number of vegetable oils, that are to be blended with HOVO, in a given bend where n can range anywhere from 1 to 10 or higher, representing blend of HOVO, such as flaxseed oil, with one or more of the other vegetable oils.

V_{Blend} is the total volume of the vegetable oil blend in ml.

ω6_{Blend} is the total omega 6 fatty acid in a given vegetable oil blend in mg per ml.

ω3_{Blend} is the total omega 3 fatty acid in a given vegetable oil blend in mg per ml.

For the purpose of obtaining vegetable oil blends that will provide ω6:ω3 fatty acid ratio, not limited to but including, of 1:1, 1:2, 2:1, 1.5:1, 1:1.5 etc., constraints (steps 103 and 104 in FIG. 1), including but not limited to the ones shown in equation (4), (4a), (4b), and (4c), are applied when solving the above equations (1) through (3) (or their variations as illustrated in this invention). For example, constraint (4a) can be used to obtain a vegetable oil blend that provides a ω6:ω3 fatty acid ratio of 1:1 {(i.e. p=1 and q=1 in equation (4)}, constraint (4b) can be used to obtain a vegetable oil blend that provides a ω6:ω3 fatty acid ratio of 2:1 {(i.e. p=2 and q=1 in equation (4)}, and constraint (4c) can be used to obtain a vegetable oil blend that provides a ω6:ω3 fatty acid ratio of 1:2 {(i.e. p=1 and q=2 in equation (4)}. Additionally, constraints limiting the amount of each of ω6 and ω3 fatty acids in the blend and the amount or percentage of each individual oil in the blend can also be applied. These and the constraints shown in equations (4), (4a), (4b) and (4c) are only exemplary and are not meant to limit the number and type of constraints that may be applied in solving equations (1) through (3) and their variations (as illustrated in the present invention), and several other combinations and permutations of constraints are possible when solving equations (1) through (3) and their variations (as illustrated in the present invention). The examples of some of such additional constraints may include but are not limited to defined values of V_{HOVO}, V_Oilᵢ, V_{Blend}, etc. The following equations (4), (4a), (4b), and (4c), where p and q are the multiplier variables having values of full integers or fractional numbers, show exemplary constraints for ω6:ω3 fatty acid ratios when solving equations (1) through (3) and their variations as illustrated in the present invention.

p×ω6_{Blend}=q×ω3_{Blend} (4)

ω6_{Blend}=ω3_{Blend}, where p=q=1 (4a)

2×ω6_{Blend}=ω3_{Blend}, where p=2and q=1 (4b)

ω6_{Blend}=2×ω3_{Blend}, where p=1and q=2 (4c)

Once a vegetable oil blend is formulated as described, the amounts of the other nutrient and fatty acid components in such a blend can be determined using, including but not limited to, such as the following equations (5a), (5b), (5c) and their variations as illustrated in the present invention, either during or after the numerical simulation/calculations are performed.

$${PUFA}_{Blend} = \frac{( {PUFA}_{HOVO} \times {V}_{HOVO} ) + {\sum }_{i = 1}^{n} ( {PUFA}_{i} \times {V\_oil}_{i} )}{{V}_{Blend}} \qquad ( 5 a )$$

$${MUFA}_{Blend} = \frac{( {MUFA}_{HOVO} \times {V}_{HOVO} ) + {\sum }_{i = 1}^{n} ( {MUFA}_{i} \times {V\_oil}_{i} )}{{V}_{Blend}} \qquad ( 5 b )$$

$$ω {9}_{Blend} = \frac{( ω {9}_{HOVO} \times {V}_{HOVO} ) + {\sum }_{i = 1}^{n} ( ω {9}_{i} \times {V\_oil}_{i} )}{{V}_{Blend}} \qquad ( 5 c )$$

These equations are embedded in the worksheet cells of last few rows of part 111, the last two columns of part 116 and worksheet cells of part 117b of FIG. 2A. Wherein,

PUFA_{HOVO} is the amount of polyunsaturated fatty acid (PUFA) content of High Omega 3 fatty acid containing Vegetable Oil (HOVO), such as flaxseed oil, in mg per ml.

MUFA_{HOVO} is the amount of monounsaturated fatty acid (MUFA) content of High Omega 3 fatty acid containing Vegetable Oil (HOVO), such as flaxseed oil, in mg per ml.

ω9_{HOVO} is the amount of omega 9 fatty acid content of High Omega 3 fatty acid containing Vegetable Oil (HOVO), such as flaxseed oil, in mg per ml.

PUFAᵢ is the amount of polyunsaturated fatty acid (PUFA) content of vegetable oil i, that is to be blended with HOVO, in mg per ml.

MUFAᵢ is the amount of monounsaturated fatty acid (MUFA) content of vegetable oil i, that is to be blended with HOVO, in mg per ml.

ω9, is the amount of omega 9 fatty acid content of vegetable oil i, that is to be blended with HOVO, in mg per ml.

PUFA_{Blend} is the total amount of polyunsaturated fatty acid (PUFA) in a given vegetable oil blend in mg per ml.

MUFA_{Blend} is the total amount of monounsaturated fatty acid (MUFA) in a given vegetable oil blend in mg per ml.

ω9_{Blend} is the total amount of omega 9 fatty acids in a given vegetable oil blend in mg per ml.

i is the number from 1 to n, each representing separate pure or individual vegetable oil (derived from a single plant or vegetable or any of their parts) that is to be blended with HOVO.

It is understood that, although not specifically described above, the present invention also enables the development of equations similar to equations (5a), (5b), and (5c) for any other additional nutrients or fatty acid components of the vegetable oils and their blends, wherein PUFA, MUFA or ω9 can be replaced with any other vegetable oil component. Thus, the above equations are not meant to limit the scope of the invention but rather provide exemplary equations and descriptions for one skilled in the art to variously employ the present invention.

Use of More than One HOVO

In certain embodiments, this invention can also be used for creating desired vegetable oil blends using the equations and the methods described in this invention where more than one High Omega 3 fatty acid containing Vegetable Oil (HOVO), such as, including but not limited to, flaxseed oil, chia seed oil, algal oil, hemp seed oil etc., can be used in the final blend to achieve a certain predetermined balance of fatty acids (omega 3, omega 6, omega 9, and other fatty acids and oil nutrients) and a certain predetermined ω6:ω3 fatty acid ratio. Additional factors and constraints, such as, including but not limited to, the percentages of oils other than HOVO desired in a blend, a certain flavor profile desired in the final blend, etc. may also be used when selecting one specific HOVO or more than one HOVO. For example, in certain embodiments, if flaxseed oil and algal oil are desired in a given blend, they will serve as HOVO₁ and HOVO₂ respectively and the remaining desired oils will be added to the summation part of equations (1) through (3) and equations (5a) through (5c). Thus, for this example, equations (1) through (3) and equations (5a) through (5c) can be written as equations (1a) through (3a) and equations (5d) through (5f) respectively as follows.

(ω6_{HOVO}_{₁}×V_{HOVO}_{₁})+(ω6_{HOVO}_{₂}×V_{HOVO}_{₂})+Σᵢ₌₁ⁿ(ω6ᵢ×V_oilᵢ)=ω6_{Blend}×V_{Blend} (1a)

(ω3_{HOVO}_{₁}×V_{HOVO}_{₁})+(ω3_{HOVO}_{₂}×V_{HOVO}_{₂})+Σᵢ₌₁ⁿ(ω3ᵢ×V_oilᵢ)=ω3_{Blend}×V_{Blend} (2a)

V_{Blend}=V_{HOVO}_{₁}+V_{HOVO}_{₂}Σᵢ₌₁ⁿ(V_Oilᵢ) (3a)

Wherein,
ω6HOVO₁ and ω6_{HOVO2} are the amount of omega 6 fatty acid content of Vegetable Oils HOVO₁ and HOVO₂, in mg per ml.
ω3HOVO₁ and ω3HOVO₂ are the amount of omega 3 fatty acid content of Vegetable Oils HOVO₁ and HOVO₂, in mg per ml.

ω6i and ω3i are the amounts of omega 6 and omega 3 fatty acid content, in mg per ml, of oil that is to be blended with HOVO₁ and HOVO₂.

V_oil₁ is the volume, in ml, of oil that is to be blended with HOVO₁ and HOVO₂.

V_{HOVO1} and V_{HOVO2} are the volumes of the Vegetable Oils HOVO₁ and HOVO₂, in the blend in ml.

Rest of the variables in the above equations (1a) through (3a) are as defined above for equations (1) through (3). Similarly, equations (5a) through (5c) can also be written as follows:

$${PUFA}_{Blend} = \frac{\begin{matrix}( {PUFA}_{{HOVO}_{1}} \times {V}_{{HOVO}_{1}} ) + \\ ( {PUFA}_{{HOVO}_{2}} \times {V}_{{HOVO}_{2}} ) + {\sum }_{i = 1}^{n} ( {PUFA}_{i} \times {V\_oil}_{i} )\end{matrix}}{{V}_{Blend}} \qquad ( 5 d )$$

$${MUFA}_{Blend} = \frac{\begin{matrix}( {MUFA}_{{HOVO}_{1}} \times {V}_{{HOVO}_{1}} ) + \\ ( {MUFA}_{{HOVO}_{2}} \times {V}_{{HOVO}_{2}} ) + {\sum }_{i = 1}^{n} ( {MUFA}_{i} \times {V\_oil}_{i} )\end{matrix}}{{V}_{Blend}} \qquad ( 5 e )$$

$$ω {9}_{Blend} = \frac{\begin{matrix}( ω {9}_{{HOVO}_{1}} \times {V}_{{HOVO}_{1}} ) + \\ ( ω {9}_{{HOVO}_{2}} \times {V}_{{HOVO}_{2}} ) + {\sum }_{i = 1}^{n} ( ω {9}_{i} \times {V\_oil}_{i} )\end{matrix}}{{V}_{Blend}} \qquad ( 5 f )$$

These equations are embedded in the worksheet cells of last few rows of part 111, the last two columns of part 116 and worksheet cells of part 117b of FIG. 2A. Wherein,

PUFA_{HOVO1} and PUFA_{HOVO2} are the amounts of polyunsaturated fatty acid (PUFA) content of vegetable oils HOVO₁ and HOVO₂ respectively, in mg per ml.

MUFA_{HOVO1} and MUFA_{HOVO2} are the amounts of monounsaturated fatty acid (MUFA) content of vegetable oils HOVO₁ and HOVO₂ respectively, in mg per ml.

ω9_{HOVO1} and ω9_{HOVO2} are the amounts of omega 9 fatty acid content of vegetable oils HOVO₁ and HOVO₂ respectively, in mg per ml.

PUFAᵢ is the amount of polyunsaturated fatty acid (PUFA) content of vegetable oil i, that is to be blended with HOVO₁ and HOVO₂, in mg per ml.

MUFAᵢ is the amount of monounsaturated fatty acid (MUFA) content of vegetable oil i, that is to be blended with HOVO₁ and HOVO₂, in mg per ml.

ω9, is the amount of omega 9 fatty acid content of vegetable oil i, that is to be blended with HOVO₁ and HOVO₂, in mg per ml.

PUFA_{Blend}, MUFA_{Blend}, and ω9_{blend} are the total amount of PUFA, MUFA and omega 9 fatty acids respectively in the vegetable oil blend, in mg per ml.

V_oilᵢ is the volume, in ml, of oil that is to be blended with HOVO₁ and HOVO₂.

V_{HOVO1} and V_{HOVO2} are the volumes of the Vegetable Oils HOVO₁ and HOVO₂, in the blend in ml.

Rest of the variables in the above equations (5d) through (5f) are as defined above for equations (5a) through (5c).

### Solution and Simulation Strategy for the Equations

The goal of the solution strategy described and presented below is in no way to admit any limitation of the scope of the solutions but to simply provide examples and embodiments for demonstration purpose and thus the applications of the above equations can go beyond the examples and embodiments demonstrated below.

The above equations (1) through (5) and their variations (as described in this invention) can be solved using several different strategies. For demonstrating the utility of the above equations in formulating desired vegetable oil blends that provide ω6:ω3 fatty acid ratio, not limited to but including, of 1:1, 1:2, 2:1, 1.5:1, 1:1.5 etc., following example embodiments will use the GRG Nonlinear Solver in Microsoft Excel (FIG. 1, step 104, FIGS. 2A and 2B), but in practice any other nonlinear solution strategy and software can be used. For solving the equations (1) through (5) several known values will be used (FIG. 1, step 102, FIG. 2A parts 108, 113, 114). For example, the values for ω6 and ω3 fatty acids and other fatty acids for the individual oils that are being blended will be known and can be used for solving the above equations. For demonstration purpose, flaxseed oil is used as the HOVO in the following examples and embodiments, but in practice any other HOVO can be used (FIG. 1, step 101). Also, for the examples and embodiments demonstrated below, three additional oils, i.e., olive oil, coconut oil and avocado oil, will be used for formulating exemplary vegetable oil blends, for demonstration purpose (FIG. 1, step 100). To enable and practice the present invention, these and any other vegetable oils can be used for formulating various vegetable oil blend possibilities. For the examples and embodiments demonstrated below, the known values for HOVO flaxseed oil and the other three oils are as shown in Tables 1 and 2 below (also shown in FIG. 2A parts 108, 113, 114). These values are obtained from various literature and commercial sources and in certain instances their average values are used for demonstration purpose.

TABLE 1
Known values for HOVO, flaxseed oil.

| Variable | Value (mg/ml) |
|---|---|
| ω6_{HOVO} | 133.73 |
| ω3_{HOVO} | 498.13 |
| ω9_{HOVO} | 170.93 |
| PUFA_{HOVO} | 633.33 |
| MUFA_{HOVO} | 172.07 |

TABLE 2
Known values for avocado, coconut and olive oil.

| | | ω6ᵢ | ω3ᵢ | ω9ᵢ | PUFAᵢ | MUFAᵢ |
|---|---|---|---|---|---|---|
| i | Vegetable oil | (mg/ml) | (mg/ml) | (mg/ml) | (mg/ml) | (mg/ml) |
| 1 | Olive oil | 91.13 | 7.13 | 665.20 | 98.20 | 681.00 |
| 2 | Coconut oil | 15.80 | 0.20 | 58.53 | 15.87 | 58.87 |
| 3 | Avocado oil | 116.93 | 8.93 | 633.60 | 125.87 | 658.53 |

The ω6 and ω3 and other fatty acid values, shown above (Tables 1 and 2) for flaxseed oil, avocado, coconut and olive oil, and similar values for other vegetable oils, can be obtained from the literature or the manufacturers of these vegetable oils or can be determined using physical and chemical analysis of the vegetable oils (FIG. 1, step 102).

In certain embodiments for the example vegetable oil blends described below, the total volume of each blend is set at 1000 ml (as shown in FIG. 2A, cell 110 and 115 and FIG. 2B, parts 118 and 118a) for demonstration purpose, but any desired volume can be used or higher and lower volumes of vegetable oil blends can also be calculated using the results obtained using 1000 ml vegetable oil blend. For solving the above equations (1) through (5) and their variations using GRG Nonlinear Solver, initial values for certain unknown variables for the iterations will need to be defined. For this purpose, the following initial values for the variables are used for each blend iteration: V_{HOVO} (for flaxseed oil), (for olive oil, coconut oil, and avocado oil) are all set equal to 100 ml when all four oils are used in the blend (FIG. 2A, parts 109 and 115). When only three oils are used for a blend, the fourth oil that is not in the blend is set equal to 0 ml for the iterations in the GRG Nonlinear Solver (FIGS. 2A and 2B, parts 108 through 122).

The equations (1), (2) and (3) above can be re-written as follows {equations (1b), (2b), and (3b)} for use in the solution strategy described here and for the exemplary blends including but not limited to the ones described below, but these are not intended to limit combinations and permutations of solution strategies and vegetable oil blend possibilities but rather serve as examples of certain embodiments.

(ω6_{HOVO}×V_{HOVO})+(ω6₁×V_oil₁)+(ω6₂×V_oil₂)+(ω6₃×V_oil₃)=ω6_{Blend}×V_{Blend} (1b)

(ω3_{HOVO}×V_{HOVO})+(ω3₁×V_oil₁)+(ω3₂×V_oil₂)+(ω3₃×V_oil₂)=ω3_{Blend}×V_{Blend} (2b)

V_{Blend}=V_{HOVO}+V_oilᵢ+V_oil₂+V_oil₃ (3b)

Wherein,

ω6₁, ω6₂, and ω6₃ are the amounts of omega 6 fatty acid content, in mg per ml, of olive oil, coconut oil, and avocado oil respectively that are to be blended with HOVO flaxseed oil.

ω3₁, ω3₂, and ω3₃ are the amounts of omega 3 fatty acid content, in mg per ml, of olive oil, coconut oil, and avocado oil respectively that are to be blended with HOVO flaxseed oil.

V_oil₁, V_oil₂, and V_oil₃ are the volumes, in ml, of olive oil, coconut oil, and avocado oil respectively that are to be blended with HOVO flaxseed oil.

Similarly, equations (5a), (5b), and (5c) can be similarly solved where i=1, 2, 3 for olive oil, coconut oil, avocado oil respectively.

Rest of the variables are as defined previously in the above equations.

Certain embodiments of the present invention include, but are not limited to the example vegetable oil blends 1 through 16 described below. In fact, several other combinations and permutations of vegetable oil blends can be formulated using the methods described in the present invention that will provide optimal balance of fatty acids and other fat/oil nutrients.

### Example Vegetable Oil Blend 1

This example blend is formulated such that it contains more than 70% olive oil with the rest being coconut oil and flaxseed oil and has ω6:ω3 fatty acid ratio of 1:1 {i.e., using constraint/equation (4a)}, with ω6 and ω3 each equal to 93.33 mg/ml or the equivalent of 1400 mg of each in one tablespoon of the final vegetable oil blend (defined as constraints in FIG. 2A, parts 117, 117a and FIG. 2B, parts 120 and 120a). Using these constraints and the known values in Tables 1 and 2, equations (1) through (3) or {(1a) through (3a) where values for HOVO₂ are zero} or equations (1b) through (3b) are solved using the GRG Nonlinear Solver in Microsoft Excel to determine the values of V_{HOVO} and V_oilᵢ for this example vegetable oil blend (FIGS. 2A and 2B, parts 108 through 122). Final values for this blend determined in this way are shown in Table 3 along with their percentage in the whole blend (auto-populated in parts 109 and 115 of FIG. 2A after calculations/simulations are completed) where the total blend volume is 1000 ml (set in part 118a, FIG. 2B) for this example.

TABLE 3
Compositions of Example Vegetable Oil Blend 1.

| Vegetable oil in | Blend | Blend |
|---|---|---|
| Example Blend 1 | composition (ml) | composition (%) |
| Olive oil | 752.81 | 75.3 |
| Coconut oil | 70.64 | 7.0 |
| Flaxseed oil | 176.56 | 17.67 |

This vegetable oil blend has ω6:ω3 fatty acid ratio of 1:1 and contains 1400 mg of each of the ω6 and ω3 (i.e., ω6_{Blend} and ω3_{Blend}, fatty acids in one tablespoon, these values will appear in part 117a of FIG. 2A. This vegetable oil also contains 8026 mg, 2803 mg, 8208 mg of ω9, PUFA and MUFA respectively, calculated using equations (5a), (5b), and (5c); these values will appear in part 117b of FIG. 2A. This exemplary blend shows that by using the methods described in the present invention, PUFA (ω6 and ω3 fatty acid) profile of olive oil can be improved such that the final blend contains majority olive oil (70% or higher) and has ω6:ω3 fatty acid ratio of 1:1 and provides 1400 mg ω3 fatty acids and maintains the ω6 fatty acid amounts of pure olive oil (i.e., about 1400 mg per tablespoon).

### Example Vegetable Oil Blend 2

This example blend is formulated such that it contains more than 70% avocado oil with the rest being coconut oil and flaxseed oil and has ω6:ω3 fatty acid ratio of 1:1 {i.e., using constraint/equation (4a)}, with ω6 and ω3 each equal to 113.33 mg/ml or the equivalent of 1700 mg of each in one tablespoon of the final vegetable oil blend (defined as constraints in FIG. 2A, parts 117, 117a and FIG. 2B, parts 120 and 120a). Using these constraints and the known values in Tables 1 and 2, equations (1) through (3) or {(1a) through (3a) where values for HOVO₂ are zero} or equations (1b) through (3b) are solved using the GRG Nonlinear Solver in Microsoft Excel to determine the values of V_{HOVO} and V_oilᵢ for this example vegetable oil blend (FIGS. 2A and 2B, parts 108 through 122). Final values for this blend determined in this way are shown in Table 4 along with their percentage in the whole blend (auto-populated in parts 109 and 115 of FIG. 2A after calculations/simulations are completed) where the total blend volume is 1000 ml (set in part 118a, FIG. 2B) for this example.

TABLE 4
Compositions of Example Vegetable Oil Blend 2.

| Vegetable oil in | Blend | Blend |
|---|---|---|
| Example Blend 2 | composition (ml) | composition (%) |
| Avocado oil | 714.06 | 71.41 |
| Coconut oil | 71.26 | 7.13 |
| Flaxseed oil | 214.68 | 21.47 |

This vegetable oil blend has ω6:ω3 fatty acid ratio of 1:1 and contains 1700 mg of each of the ω6 and ω3 (i.e., ω6_{Blend} and ω3_{Blend}) fatty acids in one tablespoon, these values will appear in part 117a of FIG. 2A. This vegetable oil also contains 7399 mg, 3405 mg, 7671 mg of ω9, PUFA and MUFA respectively, calculated using equations (5a), (5b), and (5c); these values will appear in part 117b of FIG. 2A. This exemplary blend shows that by using the methods described in the present invention, PUFA (ω6 and ω3 fatty acid) profile of avocado oil can be improved such that the final blend contains majority avocado oil (70% or higher) and has ω6:ω3 fatty acid ratio of 1:1 and provides 1700 mg ω3 fatty acids and maintains the ω6 fatty acid amounts of pure avocado oil (i.e., about 1700 mg per tablespoon).

### Example Vegetable Oil Blend 3

This example blend is formulated such that it contains four vegetable oils, avocado oil, olive oil, coconut oil, and flaxseed oil and has ω6:ω3 fatty acid ratio of 1:1 {i.e., using constraint/equation (4a)}, with ω6 and ω3 each equal to 46.67 mg/ml or the equivalent of 700 mg of each in one tablespoon of the final vegetable oil blend (defined as constraints in FIG. 2A, parts 117, 117a and FIG. 2B, parts 120 and 120a). One more constraint is used for this blend that is for coconut oil is defined to be greater than or equal to 700 ml (i.e., >=70%). Using these constraints and the known values in Tables 1 and 2, equations (1) through (3) or {(1a) through (3a) where values for HOVO₂ are zero} or equations (1b) through (3b) are solved using the GRG Nonlinear Solver in Microsoft Excel to determine the values of V_{HOVO} and for this example vegetable oil blend (FIGS. 2A and 2B, parts 108 through 122). Final values for this blend determined in this way are shown in Table 5 along with their percentage in the whole blend (auto-populated in parts 109 and 115 of FIG. 2A after calculations/simulations are completed) where the total blend volume is 1000 ml (set in part 118a, FIG. 2B) for this example.

TABLE 5
Compositions of Example Vegetable Oil Blend 3.

| Vegetable oil in | Blend | Blend |
|---|---|---|
| Example Blend 3 | composition (ml) | composition (%) |
| Avocado oil | 172.19 | 17.22 |
| Olive oil | 38.04 | 3.80 |
| Coconut oil | 700.00 | 70.00 |
| Flaxseed oil | 89.77 | 8.98 |

This vegetable oil blend has ω6:ω3 fatty acid ratio of 1:1 and contains 700 mg of each of the ω6 and ω3 (i.e., 6_{Blend} and ω3_{Blend}, fatty acids in one tablespoon, these values will appear in part 117a of FIG. 2A. This vegetable oil also contains 2861 mg, 1401 mg, 2939 mg of ω9, PUFA and MUFA respectively, calculated using equations (5a), (5b), and (5c); these values will appear in part 117b of FIG. 2A. This exemplary blend shows that by using the methods described in the present invention, PUFA (ω6 and ω3 fatty acid) profile of coconut oil can be improved such that the final blend contains majority coconut oil (70% or higher) and has ω6:ω3 fatty acid ratio of 1:1 and provides 700 mg of ω6 and ω3 fatty acids each per tablespoon of the blend.

### Example Vegetable Oil Blend 4

This example blend is formulated such that it contains four vegetable oils, avocado oil, olive oil, coconut oil, and flaxseed oil and has ω6:ω3 fatty acid ratio of 2:1 {i.e., using constraint/equation (4b)}, with ω6 and ω3 each equal to 100 mg/ml and 50 mg/ml respectively or the equivalent of 1500 mg and 750 mg of ω6 and ω3 respectively in one tablespoon of the final vegetable oil blend (defined as constraints in FIG. 2A, parts 117, 117a and FIG. 2B, parts 120 and 120a). Using these constraints and the known values in Tables 1 and 2, equations (1) through (3) or {(1a) through (3a) where values for HOVO₂ are zero} or equations (1b) through (3b) are solved using the GRG Nonlinear Solver in Microsoft Excel to determine the values of V_{HOVO} and V_oilᵢ for this example vegetable oil blend (FIGS. 2A and 2B, parts 108 through 122). Final values for this blend determined in this way are shown in Table 6 along with their percentage in the whole blend (auto-populated in parts 109 and 115 of FIG. 2A after calculations/simulations are completed) where the total blend volume is 1000 ml (set in part 118a, FIG. 2B) for this example.

TABLE 6
Compositions of Example Vegetable Oil Blend 4.

| Vegetable oil in | Blend | Blend |
|---|---|---|
| Example Blend 4 | composition (ml) | composition (%) |
| Avocado oil | 463.82 | 46.38 |
| Olive oil | 359.03 | 35.90 |
| Coconut oil | 90.27 | 9.03 |
| Flaxseed oil | 86.88 | 8.69 |

This vegetable oil blend has ω6:ω3 fatty acid ratio of 2:1 and contains 1500 mg and 750 mg of the ω6 and ω3 (i.e., ω6_{Blend} and ω3_{Blend}) fatty acids respectively in one tablespoon, these values will appear in part 117a of FIG. 2A. This vegetable oil also contains 8293 mg, 2251 mg, 8553 mg of ω9, PUFA and MUFA respectively, calculated using equations (5a), (5b), and (5c); these values will appear in part 117b of FIG. 2A. This exemplary blend shows that by using the methods described in the present invention, despite a slightly higher ω6:ω3 fatty acid ratio of 2:1 in this blend, the amount of ω6 fatty acids is maintained close to the relatively healthy pure vegetable oils available on the market (e.g., olive oil, avocado oil, canola oil etc.).

### Example Vegetable Oil Blend 5

This example blend is formulated such that it contains four vegetable oils, avocado oil, olive oil, coconut oil, and flaxseed oil and has ω6:ω3 fatty acid ratio of 1:2 {i.e., using constraint/equation (4c)}, with ω6 and ω3 each equal to 50 mg/ml and 100 mg/ml respectively or the equivalent of 750 mg and 1500 mg of ω6 and ω3 respectively in one tablespoon of the final vegetable oil blend (defined as constraints in FIG. 2A, parts 117, 117a and FIG. 2B, parts 120 and 120a). One more constraint is used for this blend that is V_oilᵢ for avocado oil is defined to be greater than or equal to 30 ml, i.e., >=3% (defined in FIG. 2B, parts 120 and 120a). Using these constraints and the known values in Tables 1 and 2, equations (1) through (3) or {(1a) through (3a) where values for HOVO₂ are zero} or equations (1b) through (3b) are solved using the GRG Nonlinear Solver in Microsoft Excel to determine the values of V_{HOVO} and V_oilᵢ for this example vegetable oil blend (FIGS. 2A and 2B, parts 108 through 122). Final values for this blend determined in this way are shown in Table 7 along with their percentage in the whole blend (auto-populated in parts 109 and 115 of FIG. 2A after calculations/simulations are completed) where the total blend volume is 1000 ml (set in part 118a, FIG. 2B) for this example.

TABLE 7
Compositions of Example Vegetable Oil Blend 5.

| Vegetable oil in | Blend | Blend |
|---|---|---|
| Example Blend 5 | composition (ml) | composition (%) |
| Avocado oil | 30.00 | 3.00 |
| Olive oil | 103.01 | 10.30 |
| Coconut oil | 668.52 | 66.85 |
| Flaxseed oil | 198.47 | 19.85 |

This vegetable oil blend has ω6:ω3 fatty acid ratio of 1:2 and contains 750 mg and 1500 mg of the ω6 and ω3 (i.e., ω6_{Blend} and ω3_{Blend}) fatty acids respectively in one tablespoon, these values will appear in part 117a of FIG. 2A. This vegetable oil also contains 2409 mg, 2253 mg, 2451 mg of ω9, PUFA and MUFA respectively, calculated using equations (5a), (5b), and (5c); these values will appear in part 117b of FIG. 2A. This exemplary blend shows that by using the methods described in the present invention, it is possible to formulate a vegetable oil blend that has an ω6:ω3 fatty acid ratio of 1:2 with low amounts of ω6 fatty acids (750 mg).

### Additional Example Vegetable Oil Blends

More example blends of flaxseed oil with olive oil, coconut oil and avocado oil are shown in the following Tables 8 and 9 for additional exemplary demonstrations. These are also determined using the above equations (1) through (5) and their variations as described in this invention. The target ω6:ω3 fatty acid ratio for these blends is 1:1 with varying amounts of ω6 and ω3 fatty acids as shown in Table 8 (defined as constraints in FIG. 2A, parts 117, 117a and FIG. 2B, parts 120 and 120a). The following blends are formulated by solving equations (1) through (3) or {(1a) through (3a) where values for HOVO₂ are zero} or equations (1b) through (3b) using the GRG Nonlinear Solver in Microsoft Excel (FIGS. 2A and 2B, parts 108 through 122) and using the constraints (a) V_{HOVO} and V_oilᵢ are all greater than or equal to 20 ml, (b) V_{Blend} is 1000 ml, (c) constraint equation (4a) holds true, and (d) ω3_{Blend} is varied from 400 mg to 1600 mg for each blend iteration as shown in Table 8 (defined as constraints in FIG. 2A, parts 117, 117a and FIG. 2B, parts 120 and 120a). Final values for the resulting blends determined in this way are shown in Table 8 in percentage in the whole blend (auto-populated values in ml in parts 109 and 115 of FIG. 2A after calculations/simulations are completed) where the total blend volume is 1000 ml (set in part 118a, FIG. 2B) for these examples. Similarly, the ω9, PUFA and MUFA values for each of the blends in Table 8 were calculated, using equations (5a), (5b), and (5c), which are shown in Table 9 below (auto-populated in part 117b of FIG. 2A). This process of calculations/simulations for formulating multiple vegetable oil blends can also be automated, if necessary, in Microsoft Excel using Excel Macro to generate the values in tables 8 and 9 instead of manually solving for each blend, or using entirely different software or calculation tool that enables such calculations/simulations and/or automation.

TABLE 8
Compositions of additional vegetable oil blends

| | | | | | Resulting omega | | | |
|---|---|---|---|---|---|---|---|---|
| | | | | | 6 and 3 fatty | | | ω6_{Blend} + |
| | | | | | acid content per | | | ω3_{Blend} |
| | | | | | tablespoon | | Resulting | total |
| Vegetable | Blend Composition (%) | | | | (tbsp) | | ω6:ω3 | per |
| Oil Blend | Olive | Coconut | Avocado | Flaxseed | ω6_{Blend} | ω3_{Blend} | Ratio in | tbsp |
| # | Oil | Oil | Oil | Oil | (mg) | (mg) | the blend | (mg) |
| Blend 6 | 3.55 | 89.22 | 2.00 | 5.23 | 400 | 400 | 1:1 | 800 |
| Blend 7 | 10.46 | 81.07 | 2.00 | 6.47 | 500 | 500 | 1:1 | 1000 |
| Blend 8 | 17.36 | 72.92 | 2.00 | 7.72 | 600 | 600 | 1:1 | 1200 |
| Blend 9 | 20.39 | 65.77 | 4.88 | 8.96 | 700 | 700 | 1:1 | 1400 |
| Blend 10 | 20.46 | 59.36 | 9.96 | 10.21 | 800 | 800 | 1:1 | 1600 |
| Blend 11 | 20.00 | 53.10 | 15.45 | 11.46 | 900 | 900 | 1:1 | 1800 |
| Blend 12 | 19.24 | 46.91 | 21.14 | 12.71 | 1000 | 1000 | 1:1 | 2000 |
| Blend 13 | 18.27 | 40.77 | 27.00 | 13.96 | 1100 | 1100 | 1:1 | 2200 |
| Blend 14 | 24.21 | 32.87 | 27.72 | 15.20 | 1200 | 1200 | 1:1 | 2400 |
| Blend 15 | 30.42 | 12.13 | 38.50 | 18.94 | 1500 | 1500 | 1:1 | 3000 |
| Blend 16 | 32.37 | 5.25 | 42.19 | 20.19 | 1600 | 1600 | 1:1 | 3200 |

TABLE 9
Other fatty acid compositions (per tablespoon) of Blends 6-16

| Vegetable | ω9_{Blend} | PUFA_{Blend} | MUFA_{Blend} |
|---|---|---|---|
| Oil Blend # | (mg) | (mg) | (mg) |
| Blend 6 | 1461.82 | 799.33 | 1483.11 |
| Blend 7 | 2111.05 | 999.70 | 2148.50 |
| Blend 8 | 2760.28 | 1200.07 | 2813.89 |
| Blend 9 | 3305.66 | 1400.45 | 3377.09 |
| Blend 10 | 3771.64 | 1600.86 | 3862.13 |
| Blend 11 | 4223.17 | 1801.26 | 4332.96 |
| Blend 12 | 4666.98 | 2001.67 | 4796.19 |
| Blend 13 | 5104.94 | 2202.08 | 5253.66 |
| Blend 14 | 5728.43 | 2402.45 | 5893.72 |
| Blend 15 | 7287.23 | 3003.63 | 7507.17 |
| Blend 16 | 7803.63 | 3204.02 | 8041.84 |

These example blends are shown here for demonstration purpose and do not intend to show any limitations for the blend possibilities but rather show representative blends out of several blend possibilities, including of several other vegetable oils.

As shown by the example blends (Blends 1 through 16) above, depending on the consumer preference and/or the ω6:ω3 fatty acid ratio desired and/or the amounts of ω6 and ω3 fatty acids desired in the blend, precise amounts of one or more High Omega 3 fatty acid containing Vegetable Oil (HOVO), such as including but not limited to flaxseed oil, can be combined with precise amounts of one or more of the other vegetable oils {e.g. olive oil, coconut oil, avocado oil, safflower oil, rapeseed (canola) oil, sunflower oil, peanut oil, sesame oil, corn oil, soybean oil, cottonseed oil, grapeseed oil, walnut oil and so on and combinations thereof}, such that several unique vegetable oil blends can be formulated with ω6:ω3 fatty acid ratio, including but not limited to, of 1:1, 1:2, 2:1, 1.5:1, 1:1.5 etc., with ω6 and ω3 fatty acids in the desirable and optimal amounts that improve the nutritional profile and potential health benefits of the resulting vegetable oil blends.

### Vegetable Oil Blends Containing a Range of Omega 6:Omega 3 Fatty Acid Ratios

The exemplary embodiments above show how the present invention can be used to generate vegetable oil blends with omega 6:omega 3 (ω6:ω3) fatty acid ratios of whole integers, including but not limited to, of 1:1, 1:2, 1:3, 2:1, 2:2, 3:1, and so on. Similarly, the present invention can also be used to generate vegetable oil blends that contain ω6:ω3 fatty acid ratios of fractional number such as, including but not limited to, of 1.1:1, 1.2:1, 1.1:1.1, 1.2:1.2, 2.1:2.1, 2.1:2.2, 1.5:1, 2.5:1, 1.5:2, 2.5:2, 1:1.5, 2:1.5, and so on. The constraint defined in equation (4) where the values of the variables p and q can be varied to include whole integers and fractional numbers to achieve the above range of ω6:ω3 fatty acid ratios. Thus, the present invention enables the generation or production of vegetable oils that will provide ω6:ω3 fatty acid ratios that can have a wide range of combinations and permutations of various such ratios as described above.

### Blending of the Vegetable Oils

The individual vegetable oils in a given vegetable oil blend, quantities and proportions of which are determined as described in the present invention (e.g., as shown in the example blends 1-16 above), can be combined together, to formulate a vegetable oil blend of the desired properties (as described in the present invention), in a vat or a container or a mixing chamber and then thoroughly mixing until uniformly blended together (parts 105, 105a, 106, 106a, 107 in FIG. 1). This process is only exemplary and is not intended to limit how the vegetable oils are mixed and blended together for the purpose of producing the vegetable oil blends and related products described in the present invention.

### Other Products Using Vegetable Oil Blends

The vegetable oils formulated in the present invention, as shown in the examples above and more of such vegetable oil blends, that will provide ω6:ω3 fatty acid ratio, not limited to but including, of 1:1, 1:2, 2:1, 1.5:1, 1:1.5, etc., with desired and optimal amounts of ω6 and ω3 fatty acids, can also be used to produce other products, including but not limited to, such as table spreads, margarines, etc., such that these other products will provide the ω6:ω3 ratio, not limited to but including, of 1:1, 1:2, 2:1, 1.5:1, 1:1.5, etc., with ω6 and ω3 fatty acids (as well as other fatty acids and vegetable oil components) in the desirable, optimal amounts that improve the nutritional profile and potential health benefits of the resulting vegetable oil blend based products.

### OTHER PUBLICATIONS

Abdelhamid A S, Brown J T J, Brainard J S, Biswas P, Thorpe G C, Moore H J, Deane K H, AlAbdulghafoor F K, Summerbell C D, Worthington H V, Song F, Hooper L. 2020. Omega-3 fatty acids for the primary and secondary prevention of cardiovascular disease. Cochrane Database Syst. Rev. 110:1158-1159.
Alvheim A R, Malde M K, Osei-Hyiaman D, Hong Lin Y, Pawlosky R J, Madsen L, Kristiansen K, Frøyland L, Hibbeln J R. 2012. Dietary linoleic acid elevates endogenous 2-A G and anandamide and induces obesity. Obesity 20:1984-1994.
Blasbalg T L, Hibbeln J R, Ramsden C E, Majchrzak S F, Rawlings R R. 2011. Changes in consumption of omega-3 and omega-6 fatty acids in the United States during the 20th century. Am. J. Clin. Nutr. 93:950-962.
Deol P, Fahrmann J, Yang J, Evans J R, Rizo A, Grapov D, Salemi M, Wanichthanarak K, Fiehn O, Phinney B, Hammock B D, Sladek F M. 2017. Omega-6 and omega-3 oxylipins are implicated in soybean oil-induced obesity in mice. Sci. Rep. 7:1-13. http://dx.doi.org/10.1038/s41598-017-12624-9.
Djuricic I, Calder P C. 2021. Beneficial outcomes of omega-6 and omega-3 polyunsaturated fatty acids on human health: An update for 2021. Nutrients 13.
Gutiérrez S, Svahn S L, Johansson M E. 2019. Effects of omega-3 fatty acids on immune cells. Int. J. Mol. Sci. 20.
Harvey K A, Walker C L, Xu Z, Whitley P, Pavlina T M, Hise M, Zaloga G P, Siddiqui R A. 2010. Oleic acid inhibits stearic acid-induced inhibition of cell growth and pro-inflammatory responses in human aortic endothelial cells. J. Lipid Res. 51:3470-3480.
Kain V, Ingle K A, Kachman M, Baum H, Shanmugam G, Rajasekaran N S, Young M E, Halade G V. 2018. Excess ω-6 fatty acids influx in aging drives metabolic dysregulation, electrocardiographic alterations, and low-grade chronic inflammation. Am. J. Physiol.—Hear. Circ. Physiol. 314:H160-H169.
Luo J, Ge H, Sun J, Hao K, Yao W, Zhang D. 2021. Associations of dietary ω-3, ω-6 fatty acids consumption with sleep disorders and sleep duration among adults. Nutrients 13.
Medeiros-De-Moraes I M, Gotçalves-De-Albuquerque C F, Kurz A R M, De Jesus Oliveira F M, Pereira de Abreu V H, Torres R C, Carvalho V F, Estato V, Bozza P T, Sperandio M, De Castro-Faria-Neto H C, Silva A R. 2018. Omega-9 oleic acid, the main compound of olive oil, mitigates inflammation during experimental sepsis. Oxid. Med. Cell. Longev. 2018.
Morales-Lázaro S L, Llorente I, Sierra-Ramírez F, López-Romero A E, Ortiz-Renteria M, Serrano-Flores B, Simon S A, Islas L D, Rosenbaum T. 2016. Inhibition of TRPV1 channels by a naturally occurring omega-9 fatty acid reduces pain and itch. Nat. Commun. 7.
Simopoulos A P. 2002. The importance of the ratio of omega-6/omega-3 essential fatty acids. Biomed. Pharmacother. 56:365-379.
Simopoulos A P. 2016. An increase in the Omega-6/Omega-3 fatty acid ratio increases the risk for obesity. Nutrients 8:1-17.
Weir N L, Nomura S O, Steffen B T, Guan W, Karger A B, Klein R, Klein B E K, Cotch M F, Tsai M Y. 2021. Associations between omega-6 polyunsaturated fatty acids, hyperinsulinemia and incident diabetes by race/ethnicity: The Multi-Ethnic Study of Atherosclerosis. J. Am. Coll. Cardiol. 77:3195-3216.

1. A method is developed that enables the formulation and the production of one or more or several vegetable oil blend(s) that consist of precise amounts of one or more High Omega 3 (ω3) fatty acid containing Vegetable Oil (HOVO), such as including but not limited to flaxseed oil, with precise amounts of one or more of the low omega 3 (ω3) and/or high omega 6 (ω6) fatty acid containing vegetable oils, and/or vegetable oil(s) containing none or very low omega 3 (ω3) and/or omega 6 (ω6) fatty acids, selected from olive oil, coconut oil, avocado oil, safflower oil, rapeseed (canola) oil, sunflower oil, peanut oil, sesame oil, corn oil, soybean oil, cottonseed oil, grapeseed oil, walnut oil and so on and combinations thereof such that the resulting oil blend provides omega 6:omega 3 (ω6:ω3) fatty acid ratio, not limited to but including, of 1:1, 1:2, 2:1, 1.5:1, 1:1.5, and more,
wherein the method offers the ability to control and modify the amounts of ω6 and ω3 fatty acids, each varying from, including but not limited to, 100 mg to 3000 mg, depending on the amounts of these fatty acids desired in the final blend while maintaining the desired ω6:ω3 fatty acid ratio in the vegetable oil blend;
wherein the blend composition is determined using the set of equations, equations (1) through (5) and their variations, the known values of fatty acids and oil nutrients such as ω6 and ω3 fatty acids for the individual oils to be blended, along with the solution and simulation strategy, including but not limited to, the one presented in this invention, along with, including but not limited to, various constraints, as described in this invention; and
wherein the vegetable oil blends of this invention are essentially formulated using either organic or virgin or extra-virgin or refined versions of the vegetable oils or combinations thereof.

2. The vegetable oil blends of claim 1 above, can be used for producing other related products, such as including but not limited to, table spreads, and margarines, etc., that will also provide ω6:ω3 fatty acid ratio, not limited to but including, of 1:1, 1:2, 2:1, 1.5:1, 1:1.5, and more, containing ω6 and ω3 fatty acids each ranging from, including but not limited to, 100 mg to 3000 mg, as well as other optimal nutrients as desired and as determined using the present invention.

3. The methods of claim 1 can be used to improve or change the fatty acid compositions of a given single and/or pure and/or blended vegetable oil.

4. The vegetable oil blends of claim 1 may also provide additional improvements in the nutritional values, health benefits and flavor profiles as a result of the changes in their overall composition enabled by this invention.

5. The products of claim 2 may provide additional improvements in the nutritional values, health benefits and flavor profile as a result of the changes in their overall composition enabled by this invention.

6. The vegetable oil blends of claim 1 will have applications in food and dietary fields as well as any other fields wherever they may be useful.

7. The products of claim 2 will have applications in food and dietary fields as well as any other fields wherever they may be useful.
